(19) United States
(12) Patent Application Publication
(10) Pub. No.: US 2016/0157875 A1
(43) Pub. Date: Jun. 9, 2016

(54) GASTROINTESTINAL DOUBLE-GRASP TISSUE FORCEPS

(71) Applicant: Dr. Behrad Ziapour, Tabriz (IR)

(72) Inventor: Behrad Ziapour, Tehran (IR)

(21) Appl. No.: 15/044,118

(22) Filed: Feb. 16, 2016

(51) Int. Cl. A61B 17/28; A61B 17/11; A61B 17/30

(52) U.S. Cl. CPC A61B 17/282; A61B 17/30; A61B 17/1114; A61B 17/2841; A61B 2017/1125; A61B 2017/1132; A61B 2017/2845; A61B 2017/00747

(57) ABSTRACT

A gastrointestinal double-grasp surgical tissue forceps has bifurcated jaws. By gripping a piece of tissue on two sides, the jaw arms fix the tissue plate between them to enable an easier and more accurate puncture, cut, needle insertion/exit or similar procedure. By contrast, when using existing tissue forceps, forces induced by such procedures can turn the tissue on a single grasp-made-pivot. By eliminating the summative effect of these turns, although they are minimal and conventionally compensated for, the gastrointestinal double-grasp forceps is expected to improve the overall speed and control of surgeons and the quality of their manual anastomoses during gastrointestinal operations.

### BACKGROUND OF THE INVENTION

During many surgical procedures, the surgeon is required to grip tissue to perform a puncture, ligation, suture or cut. Traditionally, this gripping has been accomplished using numerous types of available tissue forceps. However, these instruments grip the intended tissue at a single point, and the above procedures are then performed in the proximity of the gripped site. Therefore, the gripped point unilaterally acts as a pivot for the force of the needle, thread, scissors, or scalpel. As a result, the tissue turns on the gripped point, and a desired right angle cut or needle insertion is not possible. For years, we have compromised by accepting these tiny deviations from the ideal. However, as the field of surgery advances, there is a greater need for instruments that can perform as accurately as possible. There are a few existing fork-like forceps that can grip tissue at two points and are theoretically capable of providing two pivots to perform the above-mentioned techniques. However, these fork-like forceps, due to their structural design, are incapable of placing very close, millimeter-sized sutures, which is necessary during intestinal anastomoses. While many surgeons consider 3 to 5 mm acceptable for their intestinal anastomosis inter-suture distance, Juliet C et al., in their “Systematic review of the technique of colorectal anastomosis” in February 2013, explained that these surgeons are probably relying on their education and instinct than on scientific evidence. In the only available study, Waninger J, Kauffmann G W, Shah I A, and Farthmann E H examined the “Influence of the distance between interrupted sutures and the tension of sutures on the healing of experimental colonic anastomoses” published in Am J Surg. 1992 March; 163(3):319-23. The results of this study demonstrated that the best healing pattern during anastomosis would be achieved by moderate sutures tension and a 1.5-mm distance between sutures. The present invention is a device that is capable of facilitating such desired suturing.

### SUMMARY OF THE INVENTION

The present invention aims to provide a surgical tissue forceps that prevents even minimal turns (i.e., deflection) of the tissue during basic techniques, including suturing and cuts. The other purpose of this instrument is to provide a surgical tissue forceps that gives the surgeon more control over his or her performance. In accordance with the above goals, an embodiment of the present invention is a tweezer-like instrument with a split jaw at the distal end. Two arms of the jaw enable the surgeon to grasp two adjacent locations of the intestinal tissue in a single bite, leaving the tissue section between these two points fixed and accessible for suturing or cutting. The increased control over the surgical site increases both the speed and quality of the entire procedure.

### BRIEF DESCRIPTION OF THE DRAWINGS

FIG. 1 shows the plane view of the forceps according to one embodiment of the present invention.

FIG. 2 shows the side view of the forceps according to one embodiment of the present invention (FIG. 1).

FIG. 3 shows a perspective view of the elements and structures of FIG. 1.

FIG. 4 shows a schematic diagram on how the double grasp helps with perpendicular needle insertion compared to a single tissue grasp according to one embodiment of the present invention.

FIG. 5 shows a schematic diagram on how the double grasp guides the needle through a tissue flap in one embodiment of the present invention.

FIG. 6 shows a schematic diagram on the interface between the jaws and prongs and their preferred dimensions in one embodiment of the present invention.

### DETAILED DESCRIPTION OF THE PREFERRED EMBODIMENT

### First Embodiment

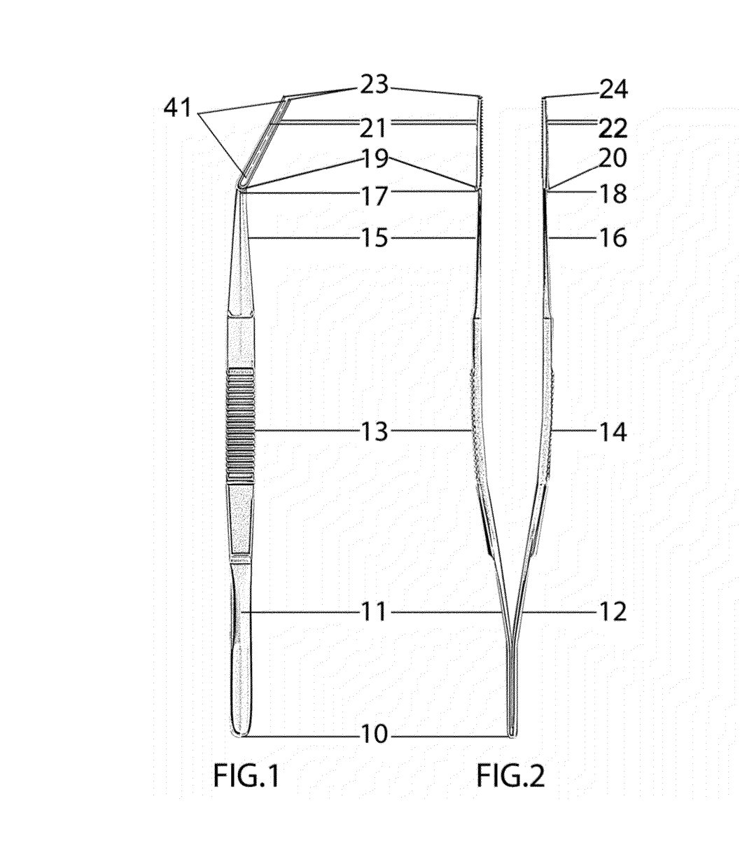

As shown in FIGS. 1 to 6, the forceps, according to one embodiment of the present invention, is a two-bladed instrument with a handle. The proximal rear ends (11 and 12) join at the back (10). The blades are resilient and biased away from one other. Slight finger compression on the opposing blades engages the jaws (19 and 20), and the prongs (21 and 22) extend from the jaws (19 and 20). The outer surfaces of the blades are knurled (13 and 14) on the middle section to make them easier for the surgeon to grasp. On this middle section, the blades diverge with a mild curvature at the proximal rear ends (11 and 12) and converge toward the distal end (15 and 16), where they finally are connected to the jaws at the blade junction (17 and 18). The obtuse angle created at the blade junction (17 and 18) with the jaws (19 and 20) in the plane view (FIG. 1) is assumed to enable ergonomic handling and a better suture field of view by deviating the surgeon's left hand leftward. In a preferred embodiment, this angle is 150°.

In one embodiment of the present invention, each opposing jaw has a pair of prongs (21 and 22). Each pair of prongs is elongated from a single jaw. The two prongs in each pair are parallel to one another, and all four single prongs have equal lengths of ten to thirty mm. The front tips of the prongs (23 and 24) are preferentially non-toothed when the forceps is intended to be used exclusively for visceral tissue.

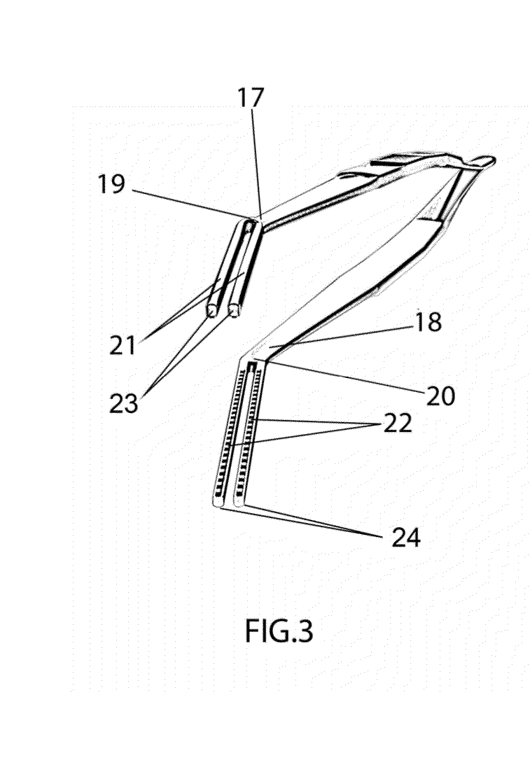

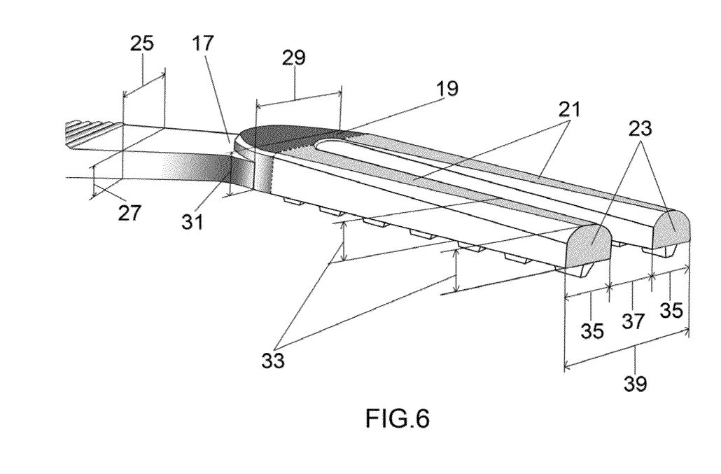

The space between the two prongs elongated from a single jaw, called the cleft (41), enables the surgeon to insert or remove a needle during the procedure, while the tissue, called the work plate, is grasped and fixed by the forceps. When gripping viable tissue, the gripping surface of prongs of the first jaw (19) aligns with the gripping surface of the prongs of the second jaw (20). To avoid any tissue damage during the double-linear grasp, in one embodiment, these inside gripping surfaces of the first and second prongs (21 and 22) have atraumatic teeth (FIG. 3) over the entire length, providing a linear grasp on either side of the work plate. The atraumatic teeth comprise a single row of pyramidal-like protrusions within the gripping surfaces. These protrusions along the entire length of each prong's gripping surface create a male-female interlocking structure with the gripping surface of the opposing prong from the other jaw. The cleft (41), as shown in FIG. 1, is the space limited by the said prongs on the sides and by the jaws at the proximal border. The cleft border at its distal end is the virtual line connecting the front tips of two prongs that are elongated from each single jaw. The distance of this distal border, as shown in FIG. 6, is d1 (37) and determines the cleft width. A cleft width is specified based on the desired distance between two consecutive sutures. In this preferred embodiment, the width of each prong is one mm; the cleft width, or d1 (37), is also one mm; and the width of the jaws is three mm.

Using any fork-like forceps, a surgeon can place only one stitch on any sinlge bite, and this ideally occurs in the midpoint of the cleft. Therefore, the shortest possible distance between two consecutive stitches cannot be less than half the width of the jaws, and the maximum jaw width must not be more than three mm if the placement of 1.5 mm distant sutures is desired.

In all previous fork-like forceps, bilateral side extensions from the junction of the handles and jaws are common key structural elements. In other words, in some existing forceps, the width of the fork-like griping section is larger than the width of the blades. These extensions act as a site in which two tines of the forceps extend in parallel and are separated.

In one embodiment of the present invention, the prongs of each jaw of the gastrointestinal double-grasp forceps lengthen along the lateral edges of the junctions (17 and 18) without any side extension. The outer width of the forceps at any point along the gripping area is constantly equal to the width of the blades at the junction (17 and 18). Therefore, a gastrointestinal double-grasp forceps has a wider blade at its junction (17 and 18) than does any existing fork-like forceps with the same cleft width. Consequently, at the junction, the gastrointestinal double-grasp forceps presents a more tolerant structure to static forces during gripping than does any fork-like forceps. This design enables industries to manufacture the new forceps with prongs close to 1 mm and capable of placing 1.5 mm consecutive sutures during gastrointestinal anastomoses. This critical need can be met when the blade junctions are still reasonably wide, up to three mm, to tolerate forces at this point. Without the aforementioned side extensions at the junctions (17 and 18), smoother maneuvers of gastrointestinal double-grasp tissue forceps in narrow anatomical spaces can also be assumed.

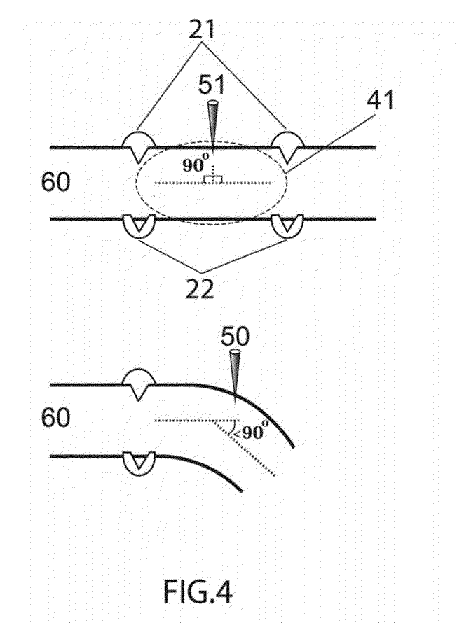

An ideal suture is theoretically formed when the needle penetrates or exits the tissue at a right angle to the tissue. As shown in FIG. 4, when using conventional tissue forceps, the needle force (50) turns the tissue (60) on a single grasp-made-pivot. However, by fixing the tissue layer between two grasp points, this embodiment of the invention ensures a perpendicular needle insertion or exit (51). This characteristic is assumed to increase the quality of the sutures.

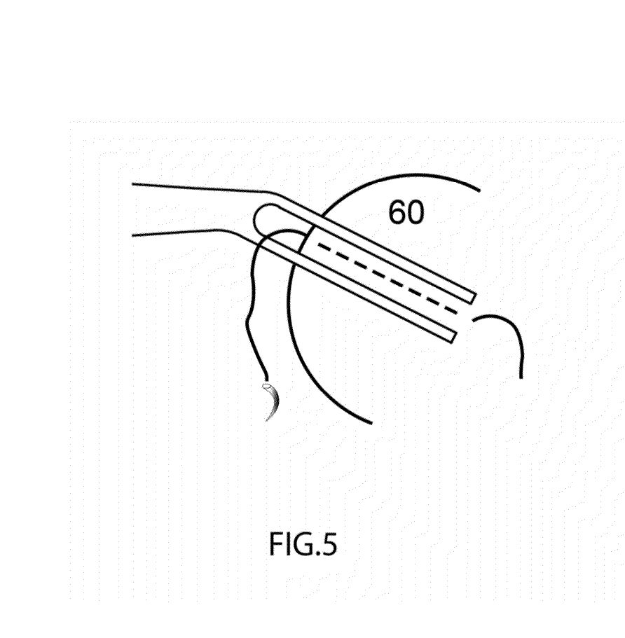

The other special characteristic of the embodiment of the present invention is flap management, as shown in FIG. 5. When part of the flap is grasped in the cleft, the needle can be much more easily driven through the tissue thickness as needed. In a further embodiment, the forceps of the present invention is used to perform precise and straight cuttings on the grasped layer of the viscera or other tissue. This is performed by grasping the tissue with the forceps and cutting it using the cutting tool in the cleft.

In one embodiment, by placing the prongs of the gastrointestinal double-grasp forceps abreast of the last stitch, the surgeon will be able to locate the exact place for the next stitch without relying on a visual estimate. By replacing the conventional subjective scale with an objective scale to place consecutive sutures, the gastrointestinal double-grasp forceps is assumed to increase both the quality and speed of gastrointestinal surgical procedures.

Because the two blades of the present forceps and their elements are mirror images of one another, each depicted dimension for the distal elements of one of two blades (FIG. 6) represents the mirror image dimensions of the opposite blade. In this preferred embodiment of the present invention, the blade width Wb (25) tapers toward the distal blade, although the blade thickness Tb (27) is constant along the distal blade. The jaws, as the extensions of the blades, have widths Wj (29) that are equal to the blade width at the junctions (17 and 18). The prongs (21 and 22) have equal widths of Wp (35). The jaw width Wj (29) and forceps tip outer width Wf (39) are both substantially equivalent to d1+(2×Wp), and d1 (37) is the aforementioned cleft width. The jaws have equal thicknesses of Tj (31), the prongs have equal thickness of Tp (33), and Tj≧Tp.

### Second Embodiment

In another embodiment of the present invention, to manufacture a gastrointestinal double-grasp tissue forceps with the option of Tj>Tp, the prongs taper with a mild slop toward their ends in the side view.

In another alternate embodiment, when a distance between two consecutive sutures of more than 1.5 mm is desired (for example, n), d1 will be corrected to d1=(2 n)−2, while the other dimensions will remain unchanged.

In the last alternate embodiment, if the forceps is used for tougher tissues, such as fascia, dura or skin, the gripping surfaces and prong tips are toothed.

In an alternate embodiment of the invention, the jaws and corresponding prongs are aligned along the blade axis. In other words, the angle at the blade junction (17 and 18) with the jaws (19 and 20) is zero.

There are many variations that can be made to the details of the invention (for example, the concept can be used for a thumb forceps or for a laparoscope forceps) without deviating from the intent and scope of the following claims, which are intended to cover all generic and specific features of the invention.

Any variations of the above techniques are also intended to be covered by this patent application.

1. A surgical forceps for gripping tissues, and gastrointestinal viscera in particular, comprising:
a first blade and a second blade with knurls on the middle sections;
a trunk formed by connecting the said first blade and the said second blade at their rear sections;
the said blades forming an acute angle at their said rear connection;
the said trunk providing a spring action at the back end that holds the grasping sections in the front apart until pressure is applied;
a first jaw and a second jaw formed by either a straight or an obtuse extension of the said first blade and the said second blade, respectively;
the first and second jaws, each include two prongs where each prong on a single jaw stands parallel to the other prong and one mm distant from it; and
gripping surfaces of the prongs of the first and second jaws such that when the front part of the forceps is clasped by manual pressure on the middle part, the gripping surface of each prong comes into complete contact with the opposing prong over its entire length.

2. The device of claim 1, including:
a cleft between two members of each pair of prongs where viable tissue is grasped linearly by the said pairs of opposing prongs on both sides.

3. The device of claim 1 to be used as an objective scale to locate the exact site for each consecutive suture (eliminating the need for surgeon's subjective visual estimate) while the said prongs are placed abreast of the last stich and the said cleft leads the needle into the correct point.

4. The device of claim 1, wherein gripping surfaces of said prongs comprise one row of atraumatic (or minimally traumatic) teeth along the full length.

5. The device of claim 4, wherein the teeth comprise protrusions that provide a male-female interlocking structure with the teeth of the gripping surface of the said opposing prongs.

6. The device of claim 4, wherein opposing prongs have a single interlocking trench-ridge tooth at their distal tips if the device is to be used for skin, dura or fascia procedures.

7. The device of claim 1, wherein the said cleft width d1 equals one mm, the said jaws have equal widths Wj, and the said prongs have equal widths Wp=1 mm, and Wj is equal to (d1+(2×Wp))=3 mm.

8. The device of claim 7, wherein the said Wj=(d1+(2 Wp)) if the said cleft width d1 equals any size other than one mm.

9. The device of claim 1, wherein said jaws have equal thicknesses Tj, said prongs have equal thicknesses Tp, Tj=Tp at the interface of the said jaws with the said prongs, and Tj>Tp as the prongs taper with a mild slop toward their ends.

10. The device of claim 4, wherein said blades at their said junction have equal thickness of Tb, equal widths of Wb, and Tj≧Tb.
